(19) United States
(12) Patent Application Publication
(10) Pub. No.: US 2022/0401716 A1
(43) Pub. Date: Dec. 22, 2022

(54) TATTOO MACHINE

(71) Applicant: CHING-HSIANG CHEN, TAINAN CITY (TW)

(72) Inventor: CHING-HSIANG CHEN, TAINAN CITY (TW)

(21) Appl. No.: 17/699,529

(22) Filed: Mar. 21, 2022

(30) Foreign Application Priority Data
Jun. 22, 2021 (TW) 110122680

(51) Int. Cl. A61M 37/00; H01F 7/10; H01F 7/08; H01F 7/16

(52) U.S. Cl. CPC A61M 37/0076; H01F 7/10; H01F 7/081; H01F 7/1615; H01F 2007/086; H01F 2007/1669; H01F 2007/163

(57) ABSTRACT

A tattoo machine includes a tubular housing and an electromagnetic assembly disposed in the tubular housing. The electromagnetic assembly includes a first through-hole extending axially therethrough. A magnet unit is disposed in the first through-hole and includes a second through-hole extending axially therethrough. A core bar extends through the second through-hole and includes a tattoo needle on an end thereof. The tattoo needle can be actuated by the magnet unit to an exposed position outside of a bottom end of the tubular housing. An electric power transmission unit is electrically connected to the electromagnetic assembly. The electric power transmission unit can be in electrical connection with a power grid or a power supply and can output alternating current to the electromagnetic assembly.

### BACKGROUND OF THE INVENTION

The present invention relates to a tattoo article and, more particularly, to a tattoo machine.

Japanese Patent Application No. P2006-527255 discloses two types of tattoo machine. FIG. 1 of this patent application illustrates a motor-type tattoo machine using a motor to actuate a tattoo needle to reciprocate vertically for achieving a tattoo effect. FIG. 2 of this patent application illustrates a coil-type tattoo machine using an electromagnetic unit which can be energized to attract an end of an iron plate downwards. In addition to actuating a tattoo needle to move downwards, the iron plate also causes the electromagnetic unit to form an open circuit and, thus, loses the magnetic force, such that the end of the iron plate cannot be attracted to move downwards. Since the iron plate is elastic due to its material characteristics, the tattoo needle can be returned to its original position. Thus, the tattoo needle can reciprocate in the vertical direction to achieve a tattoo effect.

As can be seen from FIGS. 1 and 2 of the above patent application, the tattoo machines have drawbacks. Specifically, the motor and the electromagnetic unit are not located in the movement direction of the tattoo needles. A tattoo artist holding the tattoo machine will feel that the tattoo machine tends to tilt rearwards and, thus, cannot stably hold the tattoo machine for tattooing, and a beginner tattoo artist has to spend a lot of time to adapt.

Taiwan Utility Model No. M595516 discloses a transmission mechanism for a tattoo machine. This patent integrates a motor and a tattoo needle into a pen-like configuration. Thus, a tattoo artist will scarcely feel the tilting of the tattoo machine during use. However, for a tattoo artist using the tattoo machine, it is critical to achieve a constant penetration depth of the tattoo needle regardless of the softness of the skins of various persons. However, the motor-type tattoo machine can only actuate the tattoo needle to reciprocate inflexibly. The penetration depth of the tattoo needle is shallower in the case of a thicker skin and deeper in the case of a softer skin, which is difficult to achieve the effect of constant penetration depth. Thus, improvement to the conventional tattoo machines is required.

### BRIEF SUMMARY OF THE INVENTION

In an aspect, a tattoo machine according to the present invention comprises:

a tubular housing including a hollow interior, wherein the tubular housing includes a bottom end adapted to face a human skin when in use and a top end opposite to the bottom end;

an electromagnetic assembly disposed in a section of the tubular housing, wherein the electromagnetic assembly includes a first through-hole extending axially through the electromagnetic assembly;

a magnet unit disposed in the first through-hole, wherein the magnet unit includes a second through-hole extending axially through the magnet unit;

a core bar extending through the second through-hole, wherein the core bar includes a tattoo needle on an end thereof, wherein the tattoo needle is configured to be actuated by the magnet unit to an exposed position outside of the bottom end of the tubular housing;

an elastic element disposed in the tubular housing, wherein the elastic element is directly or indirectly connected to the magnet unit or the core bar for returning the core bar; and

an electric power transmission unit electrically connected to the electromagnetic assembly, wherein the electric power transmission unit is configured to be in electrical connection with a power grid or a power supply and is configured to output pulse current to the electromagnetic assembly, such that a polarity created at a top end of the electromagnetic assembly is identical to a polarity created at a top end of the magnet unit.

Thus, by supplying pulse current to the electromagnet assembly, the polarities created at two ends of the electromagnetic assembly are identical to the polarities created at two ends of the magnet unit, thereby generating a repulsive force which can effectively actuate the core bar to thereby move the tattoo needle to an exposed position outside of the bottom end of the tubular housing. When the pulse current is at the zero voltage level, the electromagnetic assembly loses the magnetic force, such that the elastic element pushes the magnet unit, which, in turn, moves the core bar to retract the tattoo needle into the bottom end of the tubular housing. By repeating the above procedure, the tattoo needle repeatedly move in and out of the bottom end of the tubular housing, thereby achieving the tattoo effect.

Since the design of the coil-type tattoo machine is altered by the present invention, the coil-type tattoo machine can be manufactured into a pen-like configuration.

In use of the tattoo machine according to the present invention, a tattoo artist can hold it like a pen, which is extremely easy. Furthermore, the feature of a constant penetration depth of the coil-type tattoo machine is maintained. The drawbacks of the prior art are, thus, overcome.

In an example, the electromagnetic assembly includes a first electromagnetic unit, a second electromagnetic unit, and a third electromagnetic unit which are superimposed on one another. The first through-hole extending through the first electromagnetic unit, the second electromagnetic unit, and the third electromagnetic unit in sequence. The magnet unit includes a first magnet and a second magnet. The first magnet and the second magnet are superimposed on each other in the first through-hole. A polarity created at a top end of the first magnet is opposite to a polarity created at a top end of the second magnet. An intermediate section of the first magnet is located at adjoining portions of the first and second electromagnet units. An intermediate section of the second magnet is located at adjoining portions of the second and third electromagnet units.

In an example, the tattoo machine further comprises two linear bearings disposed in two end of the first through-hole, respectively. The end of the core bar and another end of the core bar extend through the two linear bearings, respectively.

In an example, the elastic element is connected to the end of the core bar. A sleeve is securely disposed in a bottom portion of the tubular housing. The core bar extends through the sleeve. The sleeve includes a first flange on an outer periphery thereof The core bar includes a second flange on an outer periphery of a bottom portion of the core bar. The elastic element is a spring having two ends respectively attached to the first flange and the second flange.

In another aspect, a tattoo machine according to the present invention comprises:

a tubular housing including a hollow interior, wherein the tubular housing includes a bottom end adapted to face a human skin when in use and a top end opposite to the bottom end;

an electromagnetic assembly disposed in a section of the tubular housing, wherein the electromagnetic assembly includes a first through-hole extending axially through the electromagnetic assembly;

a magnet unit disposed in the first through-hole, wherein the magnet unit includes a second through-hole extending axially through the magnet unit;

a core bar extending through the second through-hole, wherein the core bar includes a tattoo needle on an end thereof, wherein the tattoo needle is configured to be actuated by the magnet unit to an exposed position outside of the bottom end of the tubular housing; and

an electric power transmission unit electrically connected to the electromagnetic assembly, wherein the electric power transmission unit is configured to be in electrical connection with a power grid or a power supply and is configured to output alternating current to the electromagnetic assembly.

In an example, the electromagnetic assembly includes a first electromagnetic unit, a second electromagnetic unit, and a third electromagnetic unit which are superimposed on one another. The first through-hole extending through the first electromagnetic unit, the second electromagnetic unit, and the third electromagnetic unit in sequence. The magnet unit includes a first magnet and a second magnet. The first magnet and the second magnet are superimposed on each other in the first through-hole. A polarity created at a top end of the first magnet is opposite to a polarity created at a top end of the second magnet. An intermediate section of the first magnet is located at adjoining portions of the first and second electromagnet units. An intermediate section of the second magnet is located at adjoining portions of the second and third electromagnet units.

In an example, the tattoo machine further comprises two linear bearings disposed in two end of the first through-hole, respectively. The end of the core bar and another end of the core bar extend through the two linear bearings, respectively.

The present invention will become clearer in light of the following detailed description of illustrative embodiments of this invention described in connection with the drawings.

### DESCRIPTION OF THE DRAWINGS

FIG. 1 is a cross sectional view of a tattoo machine of an embodiment according to the present invention with a tattoo needle in a retracted position.

FIG. 2 is a cross sectional view similar to FIG. 1 with the tattoo needle moved to an exposed position.

FIG. 3 is a cross sectional view of a tattoo machine of another embodiment according to the present invention.

FIG. 4 is a cross sectional view of a tattoo machine of a further embodiment according to the present invention.

FIG. 5 is an enlarged view of a portion of FIG. 1.

### DETAILED DESCRIPTION OF THE INVENTION

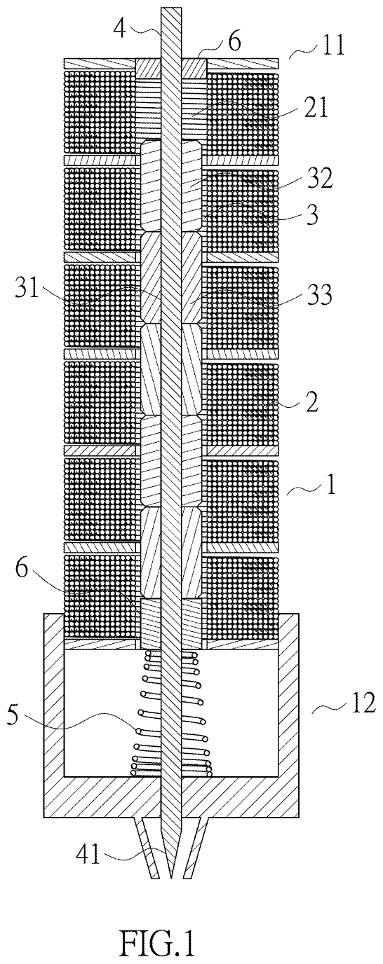

With reference to FIG. 1, a tattoo machine of an embodiment according to the present invention comprises a tubular housing 1 having a hollow interior. The tubular housing 1 includes a bottom end 12 adapted to face a human skin when in use and a top end 11 opposite to the bottom end 12. The tubular housing 1 is preferably in the form of a pen, such that a tattoo artist can easily hold the tattoo machine like a pen to easily proceed with tattooing like writing during use. Furthermore, an outer periphery of a bottom portion of the tubular housing 1 is preferably embossed to increase friction between the fingers of the tattoo artist and the tubular housing 1.

With reference to FIG. 1, the tattoo machine further comprises an electromagnetic assembly 2 disposed in a section of the tubular housing 1. The electromagnetic assembly 2 includes a first through-hole 21 extending axially through the electromagnetic assembly 2.

With reference to FIG. 1, the tattoo machine further comprises a magnet unit 3 disposed in the first through-hole 21. The magnet unit 3 includes a second through hole 31 extending axially through the magnet unit 3. The magnet unit 3 can be actuated by a magnetic force from the electromagnetic assembly 2 to move vertically in the first through-hole 21.

With reference to FIG. 1, the tattoo machine further comprises a core bar 4 extending through the second through-hole 31. The core bar 4 includes a tattoo needle 41 on an end thereof. The tattoo needle 41 is configured to be actuated by the magnet unit 3 to an exposed position outside of the bottom end 12 of the tubular housing 1. To enable the magnet unit 3 to provide better force for actuating the core bar 4, according to this embodiment shown, the electromagnetic assembly 2 preferably includes a first electromagnetic unit 22, a second electromagnetic unit 23, and a third electromagnetic unit 24 which are superimposed on one another. The first through-hole 21 extending through the first electromagnetic unit 22, the second electromagnetic unit 23, and the third electromagnetic unit 24 in sequence. Furthermore, the magnet unit 3 preferably includes a first magnet 32 and a second magnet 33. The first magnet 32 and the second magnet 33 are superimposed on each other in the first through-hole 21. A polarity of a top end of the first magnet 32 is opposite to a polarity of a top end of the second magnet 33. Furthermore, an intermediate section of the first magnet 32 is located at adjoining portions of the first and second electromagnet units 22 and 23, and an intermediate section of the second magnet 33 is located at adjoining portions of the second and third electromagnet units 23 and 24.

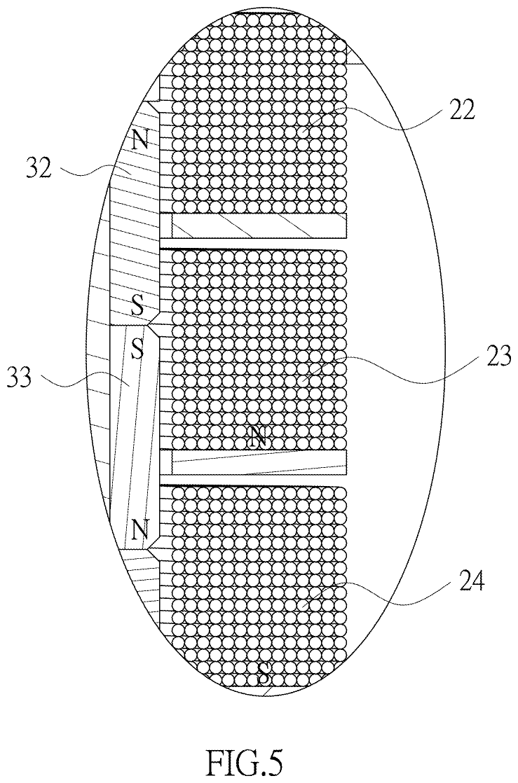

As an example, the polarities at the top end and the bottom end of the first magnet 32 are respectively north (N) polarity and south (S) polarity, whereas the polarities at the top end and the bottom end of the second magnet 33 are respectively south (S) polarity and north (N) polarity. Furthermore, the first and second magnets 32 and 33 disposed in the vertical direction are preferably secured together by an adhesive. Thus, with reference to FIG. 5, when each of the first, second, and third electromagnetic units 22, 23, and 24 is supplied with pulse direct current, the polarity created at the bottom end of the first electromagnetic unit 22 or the bottom end of the second electromagnetic unit 23 will attract the first magnet 32 to move downwards. As a result, when the magnets of the magnet unit 3 and the electromagnets of the electromagnetic assembly 2 are plural, each of the first, second, and third electromagnetic units 22, 23, and 24 will provide a downward force for an associated first or second magnet 32, 33. The sum of the forces will enable the tattoo needle 4 to be exposed in a preferred manner, providing a better tattoo effect.

According to the above structure, after supplying electricity to the electromagnetic assembly 2, the polarity created at the top end of the electromagnetic assembly 2 is identical to the polarity created at the top end of the magnet unit 3, creating a repulsive force to move the tattoo needle 4 to the exposed position outside of the bottom end 12 of the tubular housing 1. Since the tattoo needle 41 repeatedly moves out of and into the bottom end 12 of the tubular housing 1 during operation of the tattoo machine, there are two approaches of retracting the tattoo needle 41 into the bottom end 12 of the tubular housing 1.

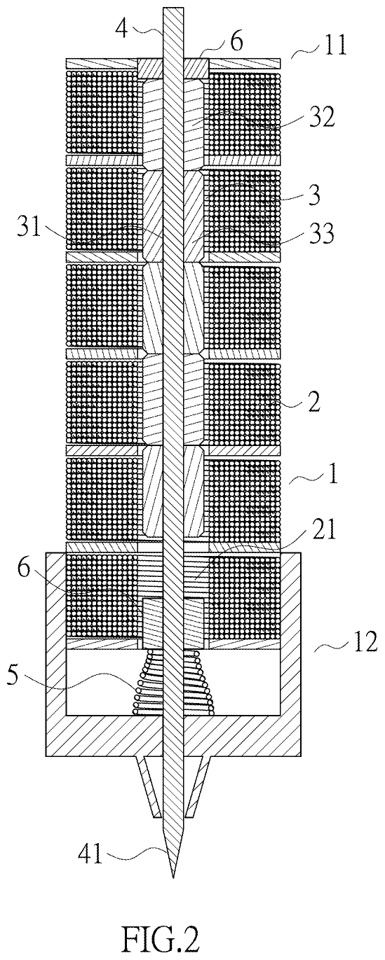

In an approach, the tattoo machine may further comprise an elastic element 5 disposed in the tubular housing 1. The elastic element 5 directly or indirectly abuts against an end of the magnet unit 3. As shown in FIGS. 1 and 2, the elastic element 5 is preferably in the form of a spring. An electric power transmission unit is electrically connected to the electromagnetic assembly 2. The electric power transmission unit is configured to be in electrical connection with a power grid or a power supply and is configured to output pulse current to the electromagnetic assembly 2, such that a polarity created at a top end of the electromagnetic assembly 2 is identical to a polarity created at a top end of the magnet unit 3. The power supply may be a battery, a dry cell, or a generator. According to this embodiment, the electric power transmission unit converts the power from the power grid or the power supply into pulse current with zero volt as the operating voltage.

Thus, when the electromagnet assembly 2 receives the pulse current, a repulsive force is created between the top end of the electromagnet assembly 2 and the top end of the magnet unit 3 due to the identical polarity thereof, moving the tattoo needle 41 to an exposed position outside of the bottom end 12 of the tubular housing 1. When the pulse current is at the zero voltage level, the electromagnetic assembly 2 loses the magnetic force, such that the elastic element 5 pushes the magnet unit 3, which, in turn, moves the core bar 4 to retract the tattoo needle 41 into the bottom end 12 of the tubular housing 1. By repeating the above procedure, the tattoo needle 41 repeatedly move in and out of the bottom end 12 of the tubular housing 1, thereby achieving the tattoo effect.

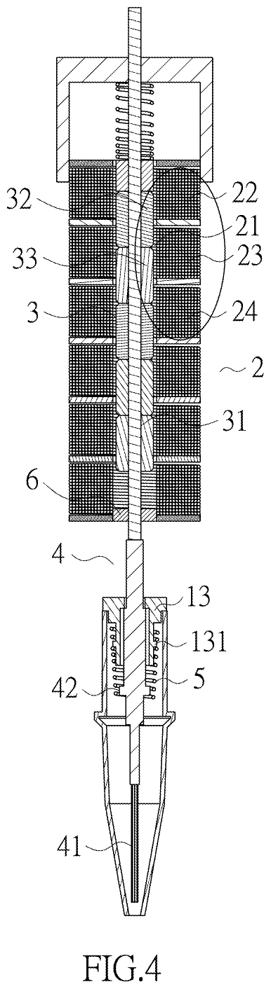

With reference to FIG. 4, in a case that the elastic element 5 is directly connected to the end of the core bar 4, a sleeve 13 is securely disposed in a bottom portion of the tubular housing 1, and the core bar 4 extends through the sleeve 13. The sleeve 13 includes a first flange 131 on an outer periphery thereof. The core bar 4 includes a second flange 42 on an outer periphery of a bottom portion of the core bar 4. The elastic element 5 is in the form of a spring having two ends respectively attached to the first flange 131 and the second flange 42. Thus, when the electromagnetic assembly 2 is supplied with direct current, after the tattoo needle 41 is moved to the exposed position, the elastic element 5 will pull the second flange 42 upwards to move the core bar 4 upwards, thereby retracting the tattoo needle 41. When the electromagnetic assembly 2 is supplied with alternating current, operation of the elastic element 5 is the same as above. In this case, the force is applied to assist in the upward movement of the core bar 4.

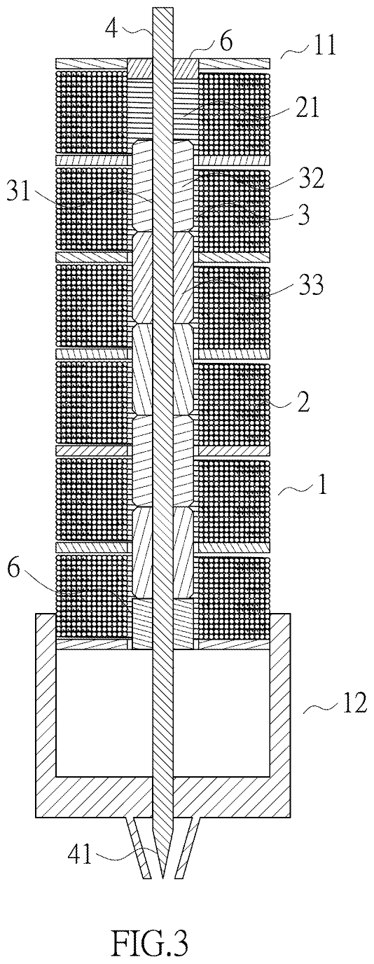

With reference to FIG. 3, another approach of retracting the tattoo needle 41 into the bottom end 12 of the tubular housing 1 is using alternative current. Specifically, the electric power transmission unit is electrically connected to the electromagnetic assembly 2 and can be electrically connected to a power grid or a power supply, supplying alternating current to the electromagnetic assembly 2. Thus, when the electromagnetic assembly 2 receives the alternating current, the top end of the electromagnetic assembly 2 alternately changes in polarity between the north (N) polarity and the south (S) polarity. A repulsive force and an attracting force are alternately generated between the electromagnetic assembly 2 and the magnet unit 3, repeatedly moving the core bar 4, thereby repeatedly moving the tattoo needle 41 out of and into the bottom end 12 of the tubular housing 1 and thereby achieving the tattoo effect. According to this embodiment, the electric power transmission unit may convert direct current from the power supply into alternating current or may output alternating current from the power grid to the electromagnetic assembly 2 for use.

The embodiments of tattoo machine according to the present invention improve the design of conventional tattoo machines, such that the coil-type tattoo machine can be manufactured into a pen-like configuration. In use of the tattoo machine according to the present invention, a tattoo artist can easily hold it like a pen, as the center of gravity is steady. The holding and operation are convenient. Furthermore, the feature of a constant penetration depth of the coil-type tattoo machine is maintained. The drawbacks of the prior art are, thus, overcome.

Furthermore, the tattoo machine of an embodiment according to the present invention may further comprise two linear bearings 6 disposed in two end of the first through-hole 21, respectively. The end of the core bar 4 and another end of the core bar 4 extend through the two linear bearings 6, respectively. The two linear bearings 6 permits smooth movement of the core bar 4 in the vertical direction while providing a positioning effect, such that the core bar 4 can be centered in the first through-hole 21.

Although specific embodiments have been illustrated and described, numerous modifications and variations are still possible without departing from the scope of the invention. The scope of the invention is limited by the accompanying claims.

What is claimed is:

1. A tattoo machine comprising:
a tubular housing including a hollow interior, wherein the tubular housing includes a bottom end adapted to face a human skin when in use and a top end opposite to the bottom end;
an electromagnetic assembly disposed in a section of the tubular housing, wherein the electromagnetic assembly includes a first through-hole extending axially through the electromagnetic assembly;
a magnet unit disposed in the first through-hole, wherein the magnet unit includes a second through-hole extending axially through the magnet unit;
a core bar extending through the second through-hole, wherein the core bar includes a tattoo needle on an end thereof, and wherein the tattoo needle is configured to be actuated by the magnet unit to an exposed position outside of the bottom end of the tubular housing;
an elastic element disposed in the tubular housing, wherein the elastic element is directly or indirectly connected to the magnet unit or the core bar for returning the core bar; and
an electric power transmission unit electrically connected to the electromagnetic assembly, wherein the electric power transmission unit is configured to be in electrical connection with a power grid or a power supply and is configured to output pulse current to the electromagnetic assembly, such that a polarity created at a top end of the electromagnetic assembly is identical to a polarity created at a top end of the magnet unit.

2. The tattoo machine as claimed in claim 1, wherein the electromagnetic assembly includes a first electromagnetic unit, a second electromagnetic unit, and a third electromagnetic unit which are superimposed on one another, wherein the first through-hole extending through the first electromagnetic unit, the second electromagnetic unit, and the third electromagnetic unit in sequence, wherein the magnet unit includes a first magnet and a second magnet, wherein the first magnet and the second magnet are superimposed on each other in the first through-hole, wherein a polarity created at a top end of the first magnet is opposite to a polarity created at a top end of the second magnet, wherein an intermediate section of the first magnet is located at adjoining portions of the first and second electromagnet units, and wherein an intermediate section of the second magnet is located at adjoining portions of the second and third electromagnet units.

3. The tattoo machine as claimed in claim 2, further comprising two linear bearings disposed in two end of the first through-hole, respectively, wherein the end of the core bar and another end of the core bar extend through the two linear bearings, respectively.

4. The tattoo machine as claimed in claim 1, wherein the elastic element is connected to the end of the core bar, wherein a sleeve is securely disposed in a bottom portion of the tubular housing, wherein the core bar extends through the sleeve, wherein the sleeve includes a first flange on an outer periphery thereof, wherein the core bar includes a second flange on an outer periphery of a bottom portion of the core bar, and wherein the elastic element is a spring having two ends respectively attached to the first flange and the second flange.

5. A tattoo machine comprising:
a tubular housing including a hollow interior, wherein the tubular housing includes a bottom end adapted to face a human skin when in use and a top end opposite to the bottom end;
an electromagnetic assembly disposed in a section of the tubular housing, wherein the electromagnetic assembly includes a first through-hole extending axially through the electromagnetic assembly;
a magnet unit disposed in the first through-hole, wherein the magnet unit includes a second through-hole extending axially through the magnet unit;
a core bar extending through the second through-hole, wherein the core bar includes a tattoo needle on an end thereof, and wherein the tattoo needle is configured to be actuated by the magnet unit to an exposed position outside of the bottom end of the tubular housing; and
an electric power transmission unit electrically connected to the electromagnetic assembly, wherein the electric power transmission unit is configured to be in electrical connection with a power grid or a power supply and is configured to output alternating current to the electromagnetic assembly.

6. The tattoo machine as claimed in claim 5, wherein the electromagnetic assembly includes a first electromagnetic unit, a second electromagnetic unit, and a third electromagnetic unit which are superimposed on one another, wherein the first through-hole extending through the first electromagnetic unit, the second electromagnetic unit, and the third electromagnetic unit in sequence, wherein the magnet unit includes a first magnet and a second magnet, wherein the first magnet and the second magnet are superimposed on each other in the first through-hole, wherein a polarity created at a top end of the first magnet is opposite to a polarity created at a top end of the second magnet, wherein an intermediate section of the first magnet is located at adjoining portions of the first and second electromagnet units, and wherein an intermediate section of the second magnet is located at adjoining portions of the second and third electromagnet units.

7. The tattoo machine as claimed in claim 6, further comprising two linear bearings disposed in two end of the first through-hole, respectively, wherein the end of the core bar and another end of the core bar extend through the two linear bearings, respectively.
